(19) United States
(12) Patent Application Publication
(10) Pub. No.: US 2008/0312534 A1
(43) Pub. Date: Dec. 18, 2008

(54) System and method for power management in a telemetric monitoring system

(76) Inventor: Kyriacos Pitsillides, Sacramento, CA (US)

Correspondence Address: Stephen B. Salai, Esq.;Harter, Secrest & Emery LLP, 1600 Bausch & Lomb Place, Rochester, NY, 14604-2711, US

(21) Appl. No.: 12/157,451

(22) Filed: Jun. 9, 2008

Related U.S. Application Data
(60) Provisional application No. 60/933,564, filed on Jun. 7, 2007.

(51) Int. Cl. A61B 8/00

(52) U.S. Cl. 600/455

(57) ABSTRACT

An implantable, multichannel pulsed-Doppler biotelemetry system is described that uses novel power management techniques to minimize power consumption to very low levels, thus, making such system suitable for long term implantation. A first power management technique described in this invention is implemented as a Closed-loop Doppler flowmeter hardware with adjustable pulse repetition rate (PRF) circuits based on a feedback circuit. Another power management technique used in the system described in this invention is implemented as a PRF-synchronized ultrasonic transducer excitation power supply. Finally, another power management technique is implemented as an adaptive sub-sampling multiplexing that enables multiple channels of flow measurement using only a single flowmeter and acquires the blood flow signal at significantly lower rates than typical Doppler flowmeters. When these techniques are incorporated in a miniature Doppler flowmeter circuit, the result is a system capable of substantial power reduction that enables this system to be used as a long-term implant.

### CROSS-REFERENCE TO RELATED APPLICATIONS

The present application claims priority under 35 USC § 119 (e) from U.S. provisional application Ser. No. 60/933,564 filed Jun. 7, 2007.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention

The present invention relates to methods of reducing power consumption of a telemetric ultrasonic device capable of measuring blood flow velocity and/or volumetric blood flow and is suitable for use as a biotelemetry sensing device. More particularly it is used in situations where the sensing device is implanted and is relying on a battery pack or other type of limited power supply.

2. Background of the Invention

In medical ultrasonic devices, ultrasonic energy is directed into the body by a transmitting piezoelectric transducer and reflected ultrasonic energy is received by the same or a separate receiving transducer.

In a common type of a medical device called the Doppler device, the frequency difference between the transmitted and received frequencies is detected. This difference in frequency is directly proportional to blood flow velocity. These Doppler devices are used most commonly to measure blood flow velocity through an artery. Such a scheme can be applied to a continuous wave (CW) and the pulsed Doppler (PD) flowmeters.

In its basic form a typical PD flowmeter consists of transmitting and receiving circuits and data processing circuits. It operates by alternating a single piezoelectric transducer between the transmitting and receiving circuits. During the transmit cycle, the piezoelectric transducer is used to insonicate an area of interest, usually a blood vessel, using a short burst of ultrasonic energy. The flowmeter then switches to the receiving cycle and the piezoelectric transducer receives the echoes generated by various structures along the ultrasonic path such as the vessel wall, red blood cell clusters, etc. The difference between the incident and reflected ultrasonic frequencies is extracted in the signal processing circuits and is referred to as the Doppler frequency shift, which is directly proportional to the blood flow velocity.

In a typical 20 MHz flowmeter, to measure a maximum Doppler frequency shift of about 20 kHz, a pulse repetition frequency (the switching rate of the piezoelectric transducer between the transmitting and receiving circuits) of about 64 kHz is used. An example of such a device is the Model 100-20 available from Triton Technologies, San Diego Calif. Other instruments that repeatedly estimate blood flow in a vessel over a period of time using one or more transducers such that a source of electrical excitation signal is applied to at least one or more transducers for an active period of a control signal sufficient to obtain a blood flow estimate and is removed from the at least one of the one or more transducers for an idle period of the control signal until a subsequent blood flow estimate is to be obtained, are described in the literature and include but are not limited to; Vessel diameter-independent volume measurements using ultrasound, C. J. Drost, Proceedings of the San Diego Biomedical symposium, v17, pp 299-302, 1978; A new ultrasonic flowmeter for intravascular application, K. G. Plass, IEEE Trans. Bio. Med. Eng., v BME-11 pp 154-156, 1964; An ultrasonic pulsed Doppler system for measuring blood flow in small vessels, C. J. Hartley and J. S. Cole, J. Appl. Physiol. V37, No. 4, pp 626-629, 1974; These manuscripts describe blood flow instruments that consume high power and are not suitable for implantable biotelemetry instrumentation.

Lately, there is a trend in medical research to use smaller animals such as rats and mice, especially the transgenic types to perform research. An important method that exists in medical research is the use of biotelemetry instruments to measure important physiological variables from research animals. Biotelemetry is a very useful tool because it enables the acquisition of data without the influence of artifacts induced by the presence of humans close to the research animals. The beneficial effects of biotelemetry are well documented in the medical literature. Therefore, a need exists for a very low power system that can measure blood flow velocity and/or volumetric blood flow. This necessitates the development of a miniature battery powered flowmeter that can operate over extended periods of time and without operator intervention. Such an instrument will provide valuable information about blood flow velocity and/or volumetric blood flow (once the internal vessel diameter is measured) in untethered biological organisms.

A manuscript titled, Totally implantable directional Doppler flowmeters, H. V. Allen et. al., Biotel. patient Monit., v6, pp 118-132, 1979” describes an instrument that was developed specifically for biotelemetry instrumentation but it is not appropriate for extended monitoring or monitoring of small vessels in small animals due to its relatively high power consumption. In addition, this design is using a high power broadband link to send the data out of the animal that limits the range to a few centimeters and is more suitable in monitoring large docile animals such as dogs.

A method to produce a low power biotelemetry blood flow velocity flowmeter is outlined in U.S. Pat. No. 5,865,749 to Doten and Brockway and pertains to a strobed ultrasonic flowmeter. This instrument achieves low power by asynchronously using power cycling or applying power on and off (strobing) to the transmit and receive circuits for a short duration, at a preset frequency of 50 or 100 Hz. Since the time that the device is on is smaller than the device is off, then a power reduction occurs. Typical on/off ratios achieved with this method are at best about 1:9 when using a 50 Hz strobing frequency and about 1:4 at worst case when using a 100 Hz strobing frequency. There are a number of disadvantages associated with this system that are addressed in this present invention:

1) The power savings advantage of the strobed system are greatly diminished when measuring blood flow in animals with high heart rates such as mice, because the time the system is on increases, while the time the system is off decreases. This means that average power consumption increases. The present invention uses a power management technique that requires substantially lower sampling times and therefore average power consumption is low.

2) The strobed flowmeter is an open loop system that does not use the output waveform to adjust operating parameters such as PRF to reduce power consumption even further. As described in this invention, this system also includes a closed-loop feedback system that automatically adjusts the PRF of the blood flowmeter to optimize the data acquisition rate of the continuously changing Doppler frequencies that occur during normal operation.

3) The strobed flowmeter uses a zero-crossing detector based on an amplitude threshold detector to convert the sinusoidal Doppler signal to square waves so that the Doppler shift frequency can be measured. A zero crossing detector is highly susceptible to mistriggering from artifacts that exceed the preset threshold. Also, when the Doppler signal is weak and below the preset threshold, then the zero crossing detector may not detect any signal crossings. The present invention utilizes a bounded slew-rate detector that only triggers an output pulse if the slew-rate of the input signal is within the range of the specified Doppler frequency. Any other artifactual frequency input that is above or below this Doppler band, such as noise spikes from nearby electromagnetic or other devices, even if it is higher in amplitude, will not trigger an output pulse. This reduces the occurrence of artifacts that could corrupt the blood flow signal quality.

Consequently, there is a need for a different power management method that can overcome the above listed shortcomings of the disclosed U.S. Pat. No. 5,865,749.

Additionally, U.S. Pat. No. 5,865,749 includes some claims that claim the totally obvious step of shutting off a circuit when not used where there is a desire or need to conserve power. This is a technique that has been known and used in electrical based systems for many years prior the filing of the application for this patent. These claims have no other distinguishing elements.

A manuscript titled, “An implantable multichannel ultrasonic pulsed Doppler blood flowmeter, Yeung, K-W, W, IEEE Eng. Med, Biol 11 Annual Conf 1989” describes a multiple channel Doppler system that uses a defined minimum time duration to sample each of the three channels described in the manuscript. This time is defined as the duration needed to sample at a pulse repetition frequency long enough to determine the mean frequencies of the Doppler shifts of the minimum bandwidth signal (ie, having the longest sampling duration) in one channel, before moving to another channel.

The method presented in this manuscript is based on an open-loop Doppler flowmeter and it does not have a means of determining the Doppler-shifted frequency of a measured sampled to estimate the minimum duration of sampling. This is introduced in the third power management method described in this invention which is based on a closed-loop Doppler flowmeter which results in substantially reduced sampling intervals for the entire range of expected Doppler-shifted waveforms, by ensuring that only the minimum number of samples is acquired.

Thus, what is needed is a more efficient system to conserve power in a systems that has a limited power supply that can overcome the deficiencies of the prior art discussed above.

### SUMMARY

Accordingly, it is the principal object of the present invention to achieve very low power consumption during the operation of a system using a power management technique based on a closed-loop Doppler flowmeter design.

An additional principal object of the present invention is to provide a circuit capable of measuring blood flow for extended periods of time using a PRF-synchronized ultrasonic transducer excitation power supply.

An additional principal object of the present invention is to provide a circuit capable of measuring blood flow for extended periods of time using an adaptive sub-sampling multiplexing technique.

A further objective of this invention is to reduce power consumption to such levels as to enable the use of a blood flowmeter in an implantable instrument that is capable of measurement of blood flow velocity and/or volumetric blood flow for long periods of time.

The present invention achieves these and other objectives by providing: various methods of power management.

The first power management technique is based on a closed-loop Doppler flowmeter with adjustable pulse repetition rate (PRF) operates by uses information obtained from the output Doppler-shifted velocity signal to obtain an estimation of an optimal adjustment of the PRF frequency to be only about 3 times higher than the expected Doppler-shifted blood flow frequency. This allows the sampling of the input signal to occur at a minimum sampling frequency while still satisfying the Nyquist minimum sampling rate criterion.

The second power management technique is based on a PRF-synchronized ultrasonic transducer excitation power supply method. This results in a conversion of the primary implant battery voltage to the higher excitation voltage used for the ultrasound transducer only when an excitation is needed. Normally, in a typical Doppler flowmeter, this voltage conversion occurs continuously. This can result in very low overall voltage conversion efficiency, and wasted power. Using the PRF-synchronized ultrasonic transducer excitation power supply technique, power waste is avoided, while still making higher voltage available to the ultrasound transducer when needed during an ultrasound transmission cycle.

The third power management technique is based on an adaptive sub-sampling multiplexing that acquires the blood flow signal at significantly lower rates than typical Doppler flowmeters. This technique provides for a method to multiplex multiple ultrasound transducers to a single-channel Doppler flowmeter, while allowing the minimum sampling duration for each channel, thus minimizing actual active periods and maximizing periods of time when the Doppler circuits are inactive. This in turn results in minimizing power consumption.

The concept of a closed-loop Doppler flowmeter allows this novel system to have knowledge of the output state and therefore make adjustments to a number of parameters. Such adjustments optimize operational parameters with the ultimate goal of maintaining complete function with the least amount of power consumption. Open-loop systems, since they lack the critical information on the status of the output state, do not have the capability to make adjustments to these operational parameters and consequently can not fully optimize the flowmeter operation.

A method for determining flow velocity of a liquid in a conduit with a variable optimal pulse repetition rate for power conservation using the steps of: a) obtaining a set of Doppler shifted velocity signals reflected from a fluid flowing in a conduit at a preset pulse repetition rate; b) determining the velocity of fluid flow from the set of Doppler shifted velocity signals generated at the preset pulse repetition rate; c) adjusting said pulse repetition rate to a lowest optimal pulse repetition rate for the velocity determined in step b; d) repeating steps a, b and c on a periodic basis with the previously determined lowest optimal pulse repetition rate as the preset pulse repetition rate in step a to thereby adjust the pulse repletion rate to a new lowest optimal pulse repetition rate for each time steps a, b and c are repeated. In a further aspect of this method each lowest optimal pulse repetition rate determined satisfies Nyquist minimum sampling rate criterion for the velocity determined. In yet another aspect of this method of this invention the first preset pulse repletion rate used in step a) when the method is commenced for the first time is the highest optimal pulse repetition rate.

In yet another aspect of this method of the invention the optimal pulse repetition rate increases with increasing velocity of the flow of fluid and decreases with decreasing velocity of the fluid flow. In yet a further aspect of the method of this invention the step of obtaining Doppler shifted velocity signals includes sensing a shift in frequency between a transmitted signal to a received reflected signal and wherein increases in frequency indicate an increase in velocity and a decrease in frequency indicate a decrease in velocity, which is used to then calculate the velocity. In yet another aspect of this method of the invention the step of obtaining Doppler shifted velocity signals includes sensing a shift in cycle length between a transmitted signal to a received reflected signal and wherein increase in the cycle indicates a decrease in velocity and a decrease in cycle indicate an increase in velocity, which is used to then calculate the velocity.

In yet another aspect of this method of the invention includes the additional step of analyzing each Doppler shifted velocity signal and rejecting it for the set of Doppler shifted velocity signals generated at the preset pulse repetition rate if the Doppler duration is deemed non valid for that particular Doppler shifted velocity signal. In yet another aspect of this invention it the step of obtaining a Doppler shifted velocity signal is accomplished with a sensor selected from a group of laser Doppler sensor, ultrasound Doppler sensor and infrared Doppler sensor. In yet another aspect of this invention the conduit is a vessel and the fluid is blood.

In yet another variation of the current invention it provides a system for determining flow characteristics of a liquid flowing in a conduit with a variable optimal pulse repetition sampling rate for power conservation having: a) a flowmeter for measuring characteristics of flow of a fluid in a conduit; b) a pulse rate frequency circuit for setting a sampling rate for the flowmeter; c) a cpu to control functioning of the flowmeter and pulse rate frequency circuit; d) a feed back circuit for said cpu to monitor changes in values of samples of flow characteristics obtained by said flowmeter; and e) wherein the cpu based on samples obtained by said flowmeter of flow characteristics adjusts a pulse repetition sampling rate of the flowmeter to an optimal pulse repetition sampling rate that minimizes samples taken and thereby conserve system power.

In a further aspect of this variation of the invention the conduit is a vessel and the fluid is blood. In yet a further aspect of this variation of the invention the characteristic being measured is fluid velocity. In yet still another aspect of this variation of the invention it includes a voltage step-up and triggering circuit controlled by the cpu that provides power to the flowmeter each time it takes a sample and is only activated during the period of time it necessary to actually generate a signal for taking a sample to thereby achieve power conservation.

In yet another aspect of this variation of the invention the optimal pulse repetition sampling rate satisfies Nyquist minimum sampling rate criterion. In still another aspect of this variation of the invention flowmeter has plurality of sensing devices for sensing flow characteristics and that measure said flow characteristics at different locations but share control, power and activation circuitry through signal multiplexing controlled by said cpu.

### BRIEF DESCRIPTION OF THE DRAWINGS

The invention will be better understood by an examination of the following description, together with the accompanying drawings, in which:

FIG. 1 is a block diagram of a preferred embodiment of Doppler flowmeter of the present invention (Abbreviations: μC—microcontroller; PRF—Pulse Repetition Frequency; Synch-HV PS—Synchronized High Voltage power Supply; USTX—Ultrasound Transmitter; USRX—Ultrasound Receiver);

FIG. 2 is a graphical representation of a preferred embodiment of the present inventions use of the feedback information to adjust the Pulse Repetition Frequency (PRF) of a Doppler flowmeter;

FIG. 3 is a flow chart one a method of the present invention;

FIG. 4 is a flow chart of another variation of the method of the present invention depicted in FIG. 3;

FIG. 5 is a timing and signal diagram showing the present inventions High voltage Power Supply (HVPS) synchronized with the Pulse Repetition Frequency (PRF) pulses and the resulting ultrasound transmitter output excitation signal (USTX);

FIG. 6 is a block diagram of the Synchronized High Voltage Power Supply circuit as used in this application;

FIG. 7 is a preferred embodiment of the Synchronized High Voltage Power Supply circuit as used in this application;

FIG. 8A is a schematic of one preferred embodiment of a portion of circuit of that might use the adaptive sub-sampling method as described in this application;

FIG. 8B a schematic of a timing signal that could control operation of a system using an Adaptive sub-sampling method; and

FIG. 9 Preferred embodiment of a biotelemetry system using the new Doppler flowmeter. The bidirectional link can be based either on Radio Frequency (RF), Infra-Red (IR), Electromagnetic (EM) or Ultrasound (US) technologies.

### DETAILED DESCRIPTION OF THE PREFERRED EMBODIMENT

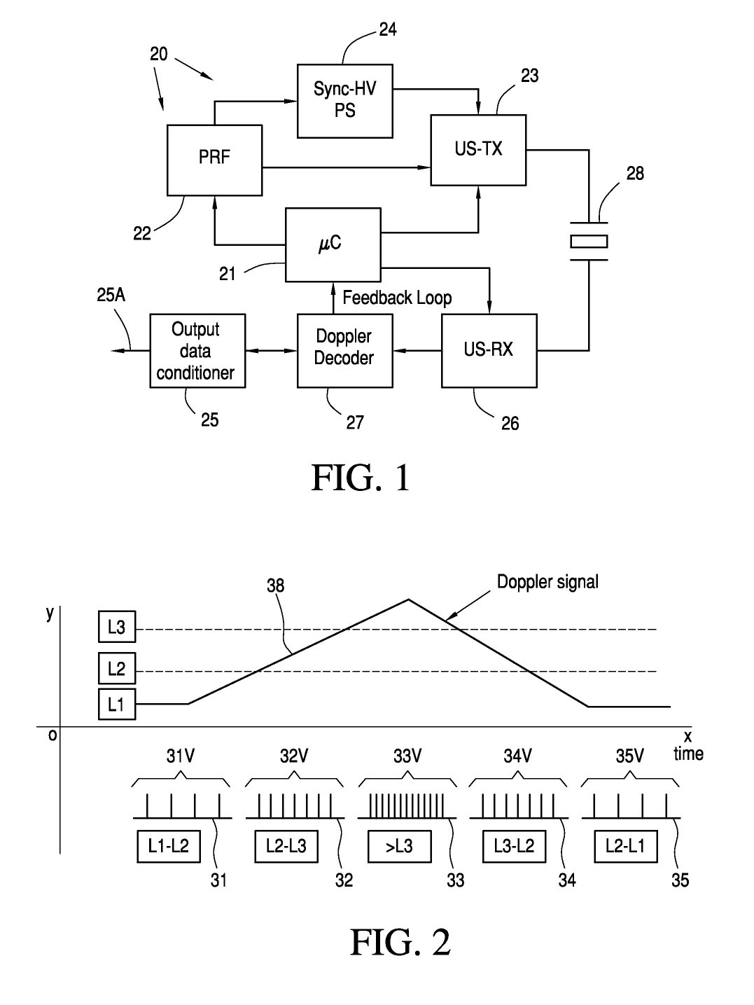

FIG. 1 shows a block diagram of the closed-loop pulsed-Doppler system 20 of the present invention with microcontroller (μC) 21, pulse repetition frequency generator (PRF) 22, ultrasound transmitter (USTX) 23, synchronized high voltage power supply (Synch-HVPS) 24, output data conditioner 25, ultrasound receiver (USRX) 26, Doppler decoder 27, and ultrasound transducer 28. The microcontroller (μC) 21 facilitates the control and measurement of all necessary functions for this system. The process is initiated by the microcontroller when it activates the pulse repetition (PRF) generator 22, which in turn activates both the Ultrasound Transmitter (USTX) 23 and the power supply (PS) 24. An added advantage of this method is the reduced side-lobe generation due to the soft-start feature of this circuit. Output data conditioner 25 connects by line 25A to a transmission unit not shown that would transmit the information on flow gathered by unit 20 to a base station at or connected to a computer for storage and processing. Unit 20 would also attach to a power supply not shown in FIG. 1.

The USTX circuit activates the piezoelectric transducer (PZT) 28 with a burst of a high frequency and high voltage signal. This causes transducer 28 to vibrate and generate an ultrasound wave. When transducer 28 is coupled to a blood vessel echoes are generated when the ultrasound wave is reflected back from clusters of red blood cells flowing in the blood vessel. These echoes or reflected ultrasound waves are received by transducer 28, amplified by the ultrasound receiver (USRX) 26 and then processed by Doppler decoder 27. The output of Doppler decoder 27 is used by the μC 21 for control purposes. The output of Doppler decoder 27 serves as an input signal to provide an estimation of the Doppler-shifted frequency measured and it is also used to control PRF generator 22, and synchronize power supply Sync-HV PS 24. The output signal is processed in block 25.

FIG. 2 shows a stream of pulses 31, 32, 33, 34 and 35, the vertical lines, generated by the PRF generator 22 with input control signal from the μC 21 which uses a closed loop feedback and look-up table to adjust the PRF generator with the appropriate pulse repetition rate for a range of input frequencies. (Each vertical line 31V, 32V, 33V, 34V and 35V rising from each horizontal line being representative of a pulse, actual pulses occur in microseconds and it would be impossible to show all that occur in their true scale.) The y-axis in FIG. 2 represents the frequency shift or change in Doppler duration of the signal that the ultrasound Doppler flowmeter system 20 (FIG. 1) senses with a changing rate in the flow of blood. The increase in the frequency shift and speed of flow going up as you move up the y-axis. The x-axis representing time period over which readings are taken. Thus Doppler signal 38 shows the actual readings of changes in frequency (Doppler duration) over time. Shifts in the Doppler signal 38 detected can vary typically from 500 Hz to 20 kHz representing blood flow of from 2 cm/sec to 200 cm/sec. The output is further conditioned using digital signal processing algorithms at circuit block 25.

The graphical representation depicted in FIG. 2 shows how the feedback information is used to adjust the Pulse Repetition Frequency (PRF) of the Doppler flowmeter. As can be seen in this simplified diagram, the system uses the information obtained by the Doppler decoder to adjust the PRF sampling rate at 3 flow velocity threshold levels (L1, L2 and L3). When these levels are crossed, the microcontroller adjusts the PRF sampling rate to be either higher or lower, and in the preferred embodiment the adjusted PRF sampling rate is dynamically adjusted to be 3 times higher than the instantaneous Doppler shifted frequency. This results in significant power savings since the system is not always at maximum PRF as in traditional Doppler flowmeters.

A preferred embodiment of the invention would assign a range of values to L1, L2 and L3 as follows: a) threshold L1 is activated when the Doppler frequency shift is between 500 Hz and 3 kHz; this would give it a sampling rate of 10 kHz with samples being taken every 100 microsec (us); b) L2 threshold is activated when the Doppler frequency is between 3 kHz and 10 kHz with a sampling rate of 32 kHz with samples being taken every 31.25 us; c) L3 threshold is activated when the frequency is over 10 kHz and less than 20 kHz; with a sampling rate of 64 kHz and samples being taken every 15.6 us. The sampling rates of L1, L2 and L3 thus meet the Nyquist requirement that an analog signal must be digitized with a sampling rate at least twice the highest frequency found in that analog signal. Thus, sampling at 3× the frequency shift gives a more faithful reproduction of the analog signal, which works well given the short acquisition durations of only 1.5 cycles of the Doppler signal. In the examples given conversion between cm/s to kHz can be done with a factor of 5.5 i.e. 100 cm/s /5.5=18.2 kHz or 5 kHz×5.5=27.5 cm/s. Although the preceding describes a preferred embodiment of the invention those skilled in the art once they have reviewed and understand the concepts of this invention will realize that a finer gradation of the sampling ranges can be done, such as L1, L2, L3, L4, etc. over the same frequency of blood flow ranges in the practice of the invention.

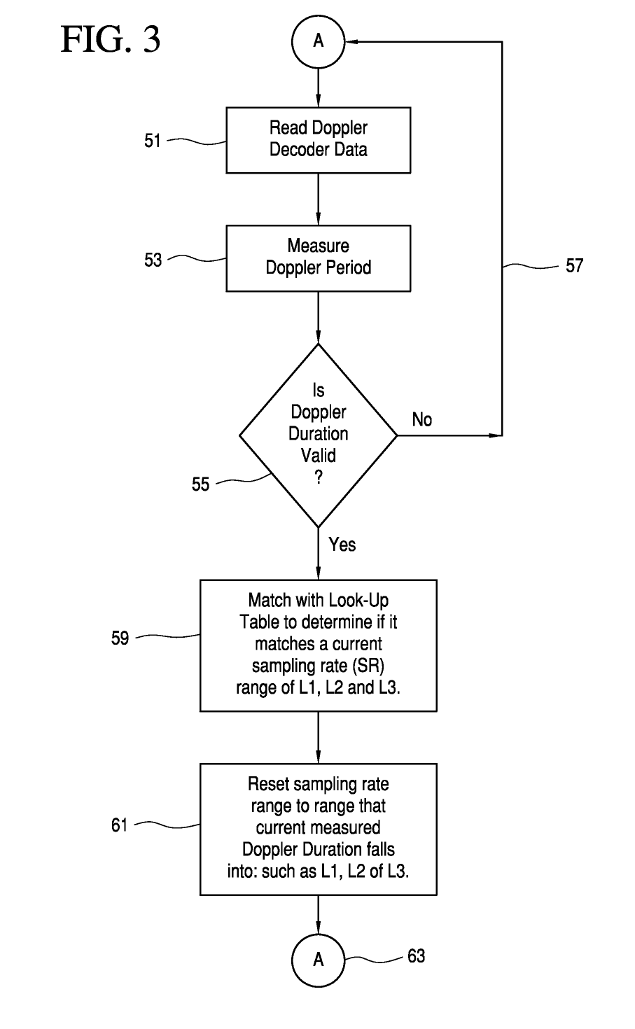

Following are two different methods for implementing the closed loop method discussed above. FIG. 3 is a flow chart showing one closed loop signal processing method of the present invention that resets the sampling rate to an optimal sampling rate. In this example the system will look at the change in the period of the reflected signal and not the change in frequency. As those skilled in the art will realize that is not a problem since the period or wave length of a signal is the inverse of the frequency: freq=1/period or wave length. In the method depicted the period or measured Doppler period is referred to at the Doppler duration or DD. In FIG. 3 with each sample reading taken it is reads the Doppler decoder data 51. The Doppler period is measured 53, it then determines of the Doppler duration measured is valid 55 if not it rejects the sample and returns to reading the next sample 57. This step is a filtering step in which spurious readings, static, noise or other corrupt signals that would interfere with proper readings are discarded. If the Doppler duration is valid it is passed along to the next step of matching the sample with a look-up table 59. At step 59 the system determines if the Doppler duration measured falls into on of the preselected sampling rates L1, L2 or L3. The system then resets 61 the sampling rate to which ever rate was identified in step 59 namely L1, L2 or L3. The system then goes back to A and starts the process all over again.

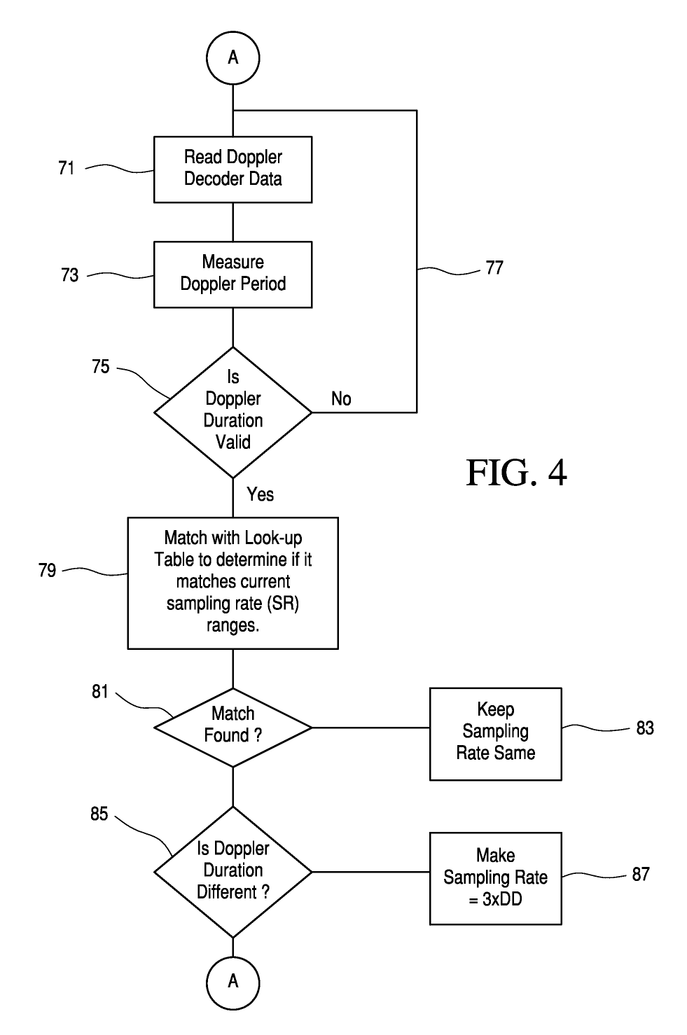

As noted above using sampling ranges L1, L2 and L3 are just one of many gradations of sampling that can be employed. Another example of the of the method of the present invention is depicted in FIG. 4 which uses a continuous range of sampling by making the sampling rate three (3) times the Doppler duration of each sample examined by this method. In FIG. 4 with each sample reading taken it reads the Doppler decoder data 71. The Doppler period is measured 73, it then determines of the Doppler duration measured is valid 75 if not it rejects the sample and returns to reading the next sample 77. This step is a filtering step in which spurious readings, static, noise or other corrupt signals interfere with proper readings. If the Doppler duration is valid it is passed along to the next step of matching the sample with a look-up table 79. At step 79 the system determines if the Doppler duration measured falls within the total sampling range. After identifying where in sampling rate range it falls in the current sampling range it determines if it is the same as the current sampling rate 81. If it is the sampling rate it is kept the same 83. If it not the same rate 85 it resets the sampling rate at 3×DD of the sample measured 87 and goes back to A to start the process over again.

The second power management technique is based on a PRF-synchronized ultrasonic transducer excitation power supply method and will now be reviewed. The present invention as noted above in a preferred embodiment would be part of biometric telemetric system used to monitor the vital functions of a biological system. Typically, it would be implanted in an animal with a battery pack and wireless transmission system to allow the animal to move about freely without human interference. It would monitor the various vital signs of the animal in this manner such as blood flow, blood pressure, etc. As noted above given the limited power supply, typically a battery pack that may have a battery with a 3 volt supply it is necessary to limit the power drawn from the battery to prolong the useful life of the battery before it has to be recharged or replaced. A typical Doppler flow meter requires a voltage source of at least 14 volts to provide the necessary excitation of an ultrasound transducer and related circuitry. Thus, a step up circuit is necessary to provide the necessary boast in available voltage. Such a circuit can quickly drain the power supply if continuously provides the boasted voltage supply. Thus, the present invention provides a system and method for limiting the actual generation of a boasted voltage supply to only those periods it is need. Generally, the boasted voltage is only needed when the USTX circuit 23 is powered to generate the ultrasound transmission.

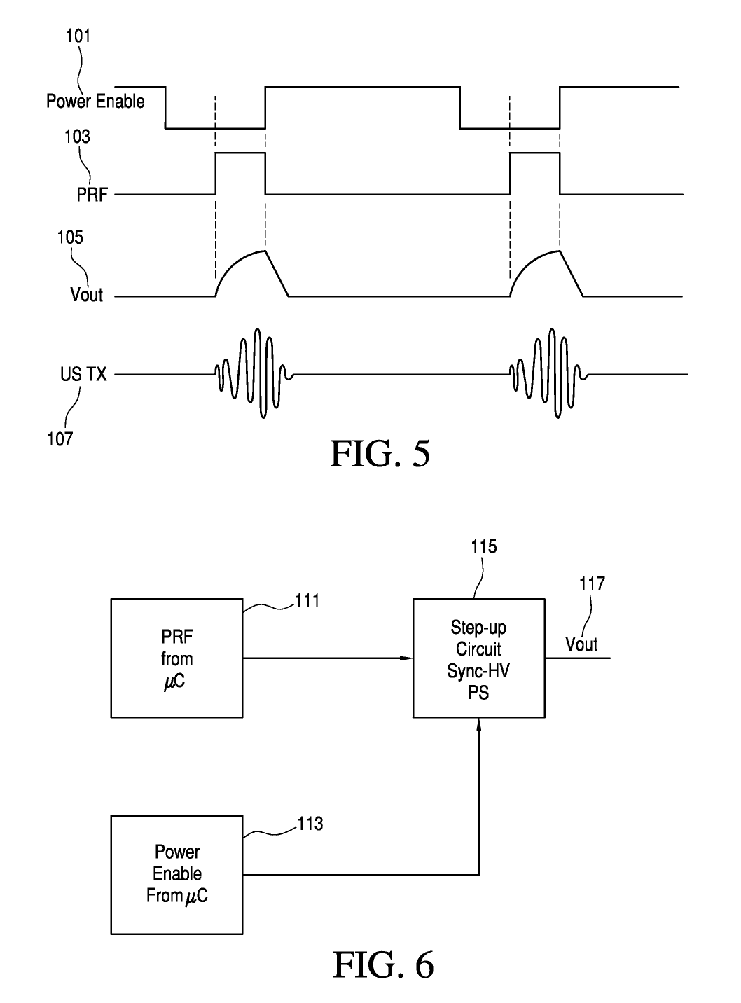

FIG. 5 is timing and signal diagram showing the High voltage Power Supply (HV-PS) synchronized with the Pulse Repetition Frequency (PRF) pulses and the resulting ultrasound transmitter output excitation signal USTX). Under control from the PRF generator 22 and the μC 21 this circuit generates a single high voltage pulse that is appropriately shaped to generate a slow-rise voltage and then a sharp exponential drop in the output voltage 105. The effect of this is shown on the output waveform 107 generated by the USTX 23. The system does this when μC 21 generates an enable signal 101. In turn enable signal from μC 21 activates PRF generator 22 to generate a PRF signal 103 to request a sample and at the same time send a signal to Sync-HV PS 24 to generate a high voltage output 105. This in turn enables USTX to generate the necessary output wave form 107. Thus, stepped up voltage is only generated when needed to thereby eliminate excessive drain on the power supply. Additionally, as shown in FIG. 5, the microcontroller 21 applies carefully measured pulses and along with the selected values in the actual circuit result in a voltage output Vout 105 that has a desired waveshape. This type of voltage waveform when applied on the Ultrasonic transducer, results in improvements in spectral purity and improved resolution by eliminating the abrupt onset and removal of the high voltage power supply waveform.

FIG. 6 is a graphic representation of the functional parts of a portion of the synchronized high power supply system namely the excitation of PRF from the micro controller 111, at the same time the micro controller sends a power enable signal to a step up circuit in Sync-high voltage power supply 115 which in turn generates the necessary voltage out put 117 to energize the USTX 23.

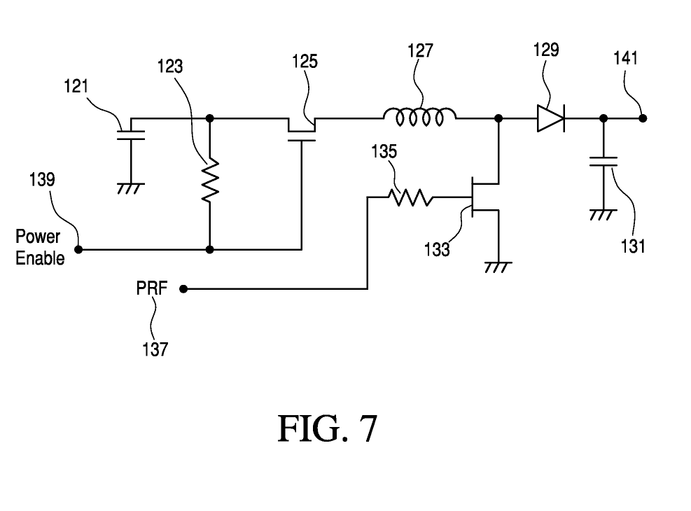

There are many types of step up-triggering circuits that could be used with the present invention. One such circuit is depicted in FIG. 7 which has capacitor 121 tied to ground, resistor 123, transistor 123, rheostat 127, diode 129, capacitor 131, transistor 133, resistor 135, PRF signal connection, power enable connection 139 and out put 141.

The third power management technique is adaptive sub-sampling multiplexing that allows the acquisition of blood flow signals at significantly lower rates than a typical Doppler flowmeter. This in part takes advantage of the first power technique described above that reduces sampling rates to a lowest optimal sampling rate necessary for acquiring data and still meeting the Nyquist criteria.

In this invention several transducers are multiplexed to a single channel Doppler flowmeter and readings are sequentially taken from each transducer in an ordered pattern. The several transducers in a standard set up would be connected to various blood vessels, usually arteries of the subject animal in which the bio-telemetric device has been implanted. The sequential readings are analyzed as described above so the optimal sampling rates described above can be set for each transducer. Actual sampling rates for each transducer may vary; depending on the feedback the system is getting from the flow in each vessel being monitored. However, since the signals from each transducer and the resulting control signals are being multiplexed, the system can easily control each transducers operation separately without any confusion.

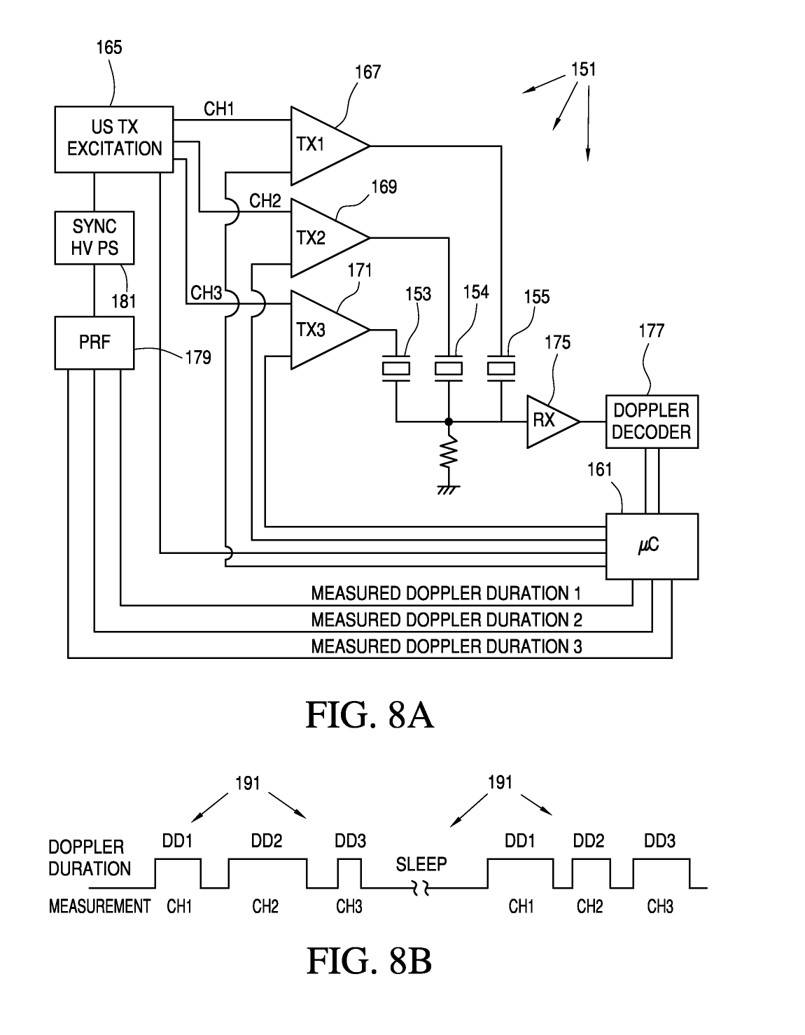

FIG. 8A provides a schematic diagram of some of the circuit components of an adaptive sub-sampling multiplexing system 151 with three transducers 153,154 and 155 which are multiplexed to a single Doppler flowmeter circuit. The entire Doppler flowmeter circuiting is not shown, just a sufficient amount to convey the concept to those skilled in the art. Micro controller 161 controls the operation of USTX (ultrasonic transmitter circuit) 165 as well as operational amplifier TX1 167 on channel one (Ch1), operational amplifier TX2 169 on channel two (Ch2) and operational amplifier TX3 171 on channel three (Ch3). Thus, it controls which of the transducers 153, 154, 155 are activated.

The signals received by each transducer are received by receiving circuit (US-RX) 175 sent to Doppler transducer 177 which in turn sends the appropriate information to micro controller 161 which in turn provides the appropriate signal on Doppler duration to pulse rate frequency module 179 which in turn provides the appropriate signal to synchronized high voltage power supply 181. Since the entire sequence is multiplexed in a prearranged pattern as indicated by excitation control Doppler duration measurement signal 191 depicted in FIG. 8B the system is able to keep track of the proper sequence of signals.

The preceding is only a rough outline of possible circuiting which could be used to practice the concepts of this aspect of the invention. It is not meant to be a complete explanation of all the circuiting or structure including transmitting of signals to the base unit which signal would also be multiplexed. However, it provides more than enough information for those skilled in the art to practice this second aspect of the present invention.

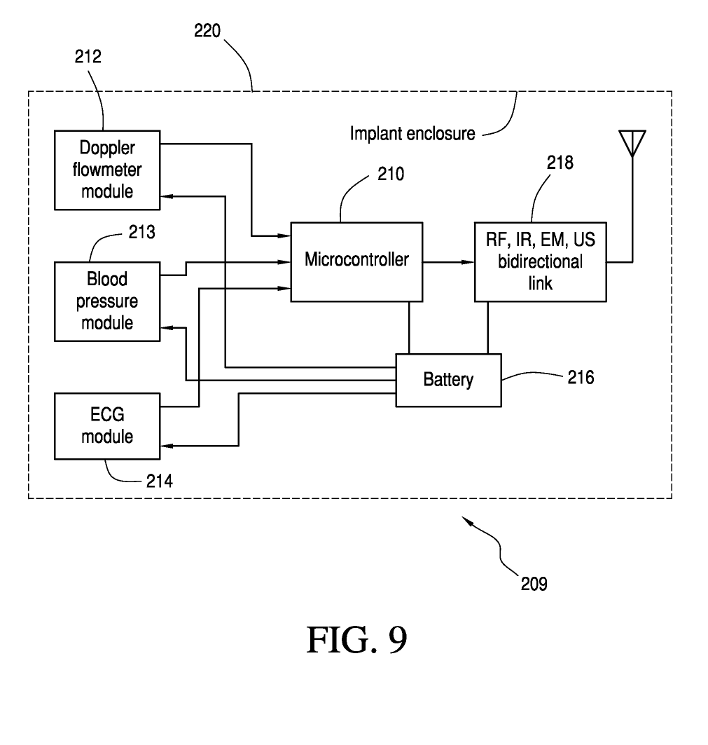

As discussed above at various points the power saving techniques and devices described would generally be implemented in bio-telemetric system used to monitor vital signs on mobile biological systems, typically animals in a research setting. FIG. 9 is a schematic diagram of one example of a preferred embodiment of a biotelemetry system 209 which could incorporate the above described invention. The major functional parts would be a microcontroller 210. Microcontroller 210 controlling Doppler flow meter 212, blood pressure module 213, ECG module 214, communication unit 218 all of which are powered by battery 216. All of these devices would be enclosed in one or more liquid impervious biocompatible packing or enclosure. The device is implantable in an animal for a significant period of time to allow monitoring of various functions of the subject animal. Communication unit 218 can use any number of various wireless systems for bidirectional communication between implantable unit 209 and a base station, not shown. Such standard wireless communication can be established with various electromagnetic, radio frequency or ultra sound based systems. These systems could include bluetooth, infrared or other radio frequency or electromagnetic wave based systems.

While the present application discusses use of an ultrasound Doppler system for determining flow of a fluid in a conduit such as blood in a vessel such as an artery once those familiar with those skilled in the art become familiar with the various power saving inventions disclosed above they will see how the concepts of the present invention can be applied to other devices that measure flow such as laser Doppler systems, infrared, electromagnetic systems without departing from the concepts of the present invention.

While the invention has been particularly shown and described with reference to a preferred embodiment thereof, it will be understood by those skilled in the art that various changes in form and detail may be made to it without departing from the spirit and scope of the invention.

I claim:

1. A method for determining flow velocity of a liquid in a conduit with a variable optimal pulse repetition rate for power conservation comprising the steps of:
a. obtaining a set of Doppler shifted velocity signals reflected from a fluid flowing in a conduit at a preset pulse repetition rate;
b. determining the velocity of fluid flow from the set of Doppler shifted velocity signals generated at the preset pulse repetition rate;
c. adjusting said pulse repetition rate to a lowest optimal pulse repetition rate for the velocity determined in step b;
d. repeating steps a, b and c on a periodic basis with the previously determined lowest optimal pulse repetition rate as the preset pulse repetition rate in step a to thereby adjust the pulse repletion rate to a new lowest optimal pulse repetition rate for each time steps a, b and c are repeated.

2. The method of claim 1 wherein each lowest optimal pulse repetition rate determined satisfies Nyquist minimum sampling rate criterion for the velocity determined.

3. The method of claim 1 were in the first preset pulse repletion rate used in step a when the method is commenced for the first time is the highest optimal pulse repetition rate.

4. The method of claim 1 wherein the optimal pulse repetition rate increases with increasing velocity of the flow of fluid and decreases with decreasing velocity of the fluid flow.

5. The method of claim 1 where the step of obtaining Doppler shifted velocity signals comprises sensing a shift in frequency between a transmitted signal to a received reflected signal and wherein increases in frequency indicate an increase in velocity and a decrease in frequency indicate a decrease in velocity, which is used to then calculate the velocity.

6. The method of claim 1 where the step of obtaining Doppler shifted velocity signals comprises sensing a shift in cycle length between a transmitted signal to a received reflected signal and wherein increase in the cycle indicates a decrease in velocity and a decrease in cycle indicate an increase in velocity, which is used to then calculate the velocity.

7. The method of claim 1 including the additional step of analyzing each Doppler shifted velocity signal and rejecting it for the set of Doppler shifted velocity signals generated at the preset pulse repetition rate if the Doppler duration is deemed non valid for that particular Doppler shifted velocity signal.

8. The method of claim 1 wherein the step of obtaining a Doppler shifted velocity signal is accomplished with a sensor selected from a group of laser Doppler sensor, ultrasound Doppler sensor and infrared Doppler sensor.

9. The method of claim 1 wherein the conduit is a vessel and the fluid is blood.

10. The method of claim 1 including the additional step of only activating a power supply to activate a transducer to generate a signal to produce Doppler shifted velocity signals.

11. The method of claim 1 including the additional step of only powering a transducer during the period of time necessary to generate a signal to obtain a reflected Doppler shifted velocity signals.

12. A system for determining flow characteristics of a liquid flowing in a conduit with a variable optimal pulse repetition sampling rate for power conservation comprising:
a) a flowmeter for measuring characteristics of flow of a fluid in a conduit;
b) a pulse rate frequency circuit for setting a sampling rate for the flowmeter;
c) a cpu to control functioning of the flowmeter and pulse rate frequency circuit;
d) a feed back circuit for said cpu to monitor changes in values of samples of flow characteristics obtained by said flowmeter; and
e) wherein the cpu based on samples obtained by said flowmeter of flow characteristics adjusts a pulse repetition sampling rate of the flowmeter to an optimal pulse repetition sampling rate that minimizes samples taken and thereby conserve system power.

13. The system of claim 12 wherein the conduit is vessel and the fluid is blood.

14. The system of claim 12 wherein the characteristic being measured is fluid velocity.

15. The system of claim 12 including a voltage step-up and triggering circuit controlled by said cpu that provides power to the flowmeter each time it takes a sample and is only activated during the period of time it necessary to actually generate a signal for taking a sample to thereby achieve power conservation.

16. The system of claim 12 wherein said optimal pulse repetition sampling rate satisfies Nyquist minimum sampling rate criterion.

17. The system of claim 12 wherein said flowmeter has plurality of sensing devices for sensing flow characteristics and that measure said flow characteristics at different locations but share control, power and activation circuitry through signal multiplexing controlled by said cpu.
